(19) United States
(12) Patent Application Publication
(10) Pub. No.: US 2008/0177333 A1
(43) Pub. Date: Jul. 24, 2008

(54) Adjustable jacking implant

(75) Inventor: Joe W. Ferguson, Collierville, TN (US)

Correspondence Address: Medtronic;Spinal and Biologics, 2600 Sofamor Danek Drive, Memphis, TN, 38132, US

(73) Assignee: Warsaw Orthopedic, Inc.

(21) Appl. No.: 11/585,387

(22) Filed: Oct. 24, 2006

(51) Int. Cl. A61B 17/56

(52) U.S. Cl. 606/304; 606/320

(57) ABSTRACT

An adjustable jacking implant useful in orthopedic surgery and associated methods are disclosed. The implant may be used to adjust the position of bones or other structures with respect to one another. In one embodiment, the implant may include a jack base, a jacking segment and a compressible spacer or sleeve. The implant may be inserted through a single stab or puncture entry. Jack base is placed in a first portion of a vertebra via a hole in an adjacent vertebra, in one embodiment, and the jacking segment is placed in the adjacent vertebra and connected to the base. After being connected, the depth of the adjustable portion of jacking segment in its vertebra determines the spacing between portions of the adjacent vertebrae.

The present disclosure relates to implants useful in orthopedic surgery. The implants may be useful for correction of spinal injuries or deformities and more specifically, but not exclusively, for maintaining a distance between bones or other semi-rigid tissues.

In the realm of orthopedic surgery, implants may be used to adjust the position of bones or other structures with respect to one another and to set these positions. For example, in the field of spinal treatment, disease, injury, or malformation can result in improper function or damage to laminae, spinous processes, articular processes, facets or other parts of one or more vertebrae can be damaged. In many such cases, the vertebrae may be unable to properly articulate or align with one another, resulting in scoliosis or other types of misaligned anatomy, pain or discomfort, and/or loss of mobility.

Many types of implants and treatments have been proposed for adjusting, repairing or otherwise treating vertebrae so as to correct vertebral damage or decrease or eliminate associated discomfort. Such implants and treatments can involve substantial surgical effort, with open surgical access, retraction of tissue, preparation and/or manipulation of vertebrae, placement and adjustment of an implant, and closing the surgical site with associated re-placement of tissues. Implants and methods for more easily changing spacing or otherwise adjusting vertebrae remain in need.

### SUMMARY

Among other things, there is disclosed an adjustable medical implant for use between portions of adjacent vertebrae having a jack base including an externally threaded anchoring portion adapted for insertion into at least a portion of a first vertebra and a central longitudinal aperture, and a jacking segment including a body portion and a shaft portion, the body portion having an externally threaded portion for insertion into at least a portion of a second vertebra adjacent said first vertebra so that its shaft portion is at least partially within the base's aperture. The jacking segment can rotate independently of the base, and turning the jacking segment relative to the base operates to alter a distance between the first and second vertebrae. A spacer member, which may be a compressible sleeve, may be located around the shaft and between the jacking segment and the base, and may be firmly attached to the body portion of the jacking segment or the base. The shaft can include a flange portion that extends substantially perpendicular to the longitudinal axis of the shaft, and that flange may abut the base when a portion of the shaft is within the aperture of the base. The body portion of the jacking segment can include an internal driving print, the aperture in the base can extend through the entire length of the base, and a portion of the aperture can have an internal driving print. The shaft portion and the body portion of the jack segment may be monolithic, and the external threads of the base and the jacking segment may be substantially identical in configuration.

There is also disclosed an adjustable implant for use between portions of adjacent vertebrae having a jack base with an externally threaded anchoring portion adapted to engage at least a portion of a first vertebra, a jacking segment having a shaft portion and a body portion that is externally threaded for engaging at least a portion of a second vertebra and having an internal driving print, and a sleeve member having a first end and a second end, wherein the base has a head surface abutting the sleeve member, and the shaft portion of the jacking segment extends through the sleeve. An axial cannula including a driving print may be in the base, and that cannula may extend through the entire length of the base. The shaft can include a flange portion extending substantially perpendicular to the longitudinal axis of the shaft, and that flange portion may abuts the base when the shaft extends into the cannula of the base. The sleeve member can be compressible and/or firmly attached to one of the base and the jacking segment.

Methods of inserting an adjustable implant between first and second adjacent vertebrae are also disclosed, among which may include accessing a patient's spine via a stab or puncture entry; making a hole in and through a portion the first and second vertebrae via the entry; inserting a jack base having an aperture through the hole in the first vertebra and into the hole in the second vertebra, so that the base is attached only to the second vertebra; inserting a jacking segment including an externally threaded body portion and a shaft portion into at least a portion of the hole in the first vertebra so that the shaft portion enters the aperture in the base and contacts a portion of the base; and turning the jacking segment with respect to the base, thereby changing the spacing between at least a portion of the vertebrae. The base may include a threaded exterior, and a base-inserting step can include threading the base through the hole in the first vertebra and into the hole in the second vertebra. A spacer element having a central aperture may be connected to the jacking segment so that the shaft extends through the central aperture of the spacer element, and the jacking-segment-inserting step may include abutting a portion of the shaft with the base. A spacer element having a central aperture may be connected to the base, and the jacking-segment-inserting step can include inserting the shaft through the central aperture of the spacer element.

### BRIEF DESCRIPTION OF THE DRAWINGS

FIG. 1 is a perspective view of an embodiment of an adjustable jacking implant.

FIG. 2 is a side elevational view of a first part of the embodiment illustrated in FIG. 1.

FIG. 3 is a top plan view of the part illustrated in FIG. 2.

FIG. 4 is a cross-sectional view of the part illustrated in FIG. 2, taken along the lines 4-4 in FIG. 3 and viewed in the direction of the arrows.

FIG. 5 is a side elevational view of a second part of the embodiment illustrated in FIG. 1.

FIG. 6 is a top plan view of the part illustrated in FIG. 5.

FIG. 7 is a cross sectional view of the part illustrated in FIG. 5, taken through line 7-7 in FIG. 6 and viewed in the direction of the arrows.

FIG. 8 is a perspective view of an embodiment as in FIG. 1 shown between adjacent vertebrae, with portions of the vertebrae cut away.

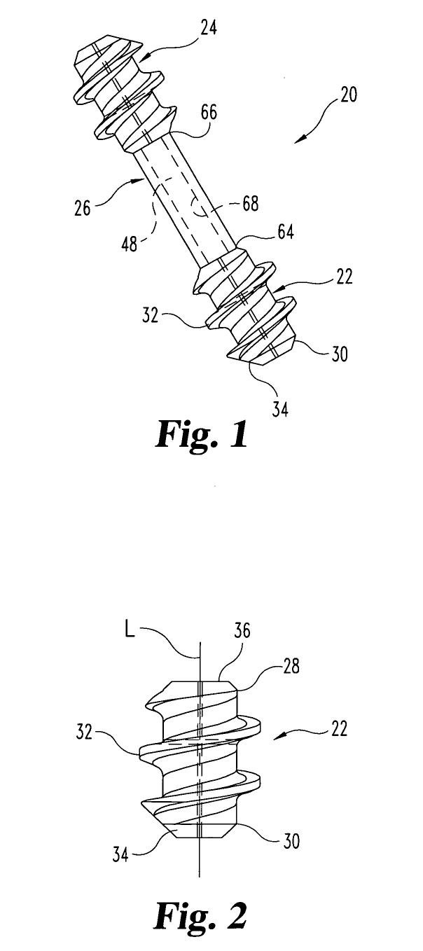

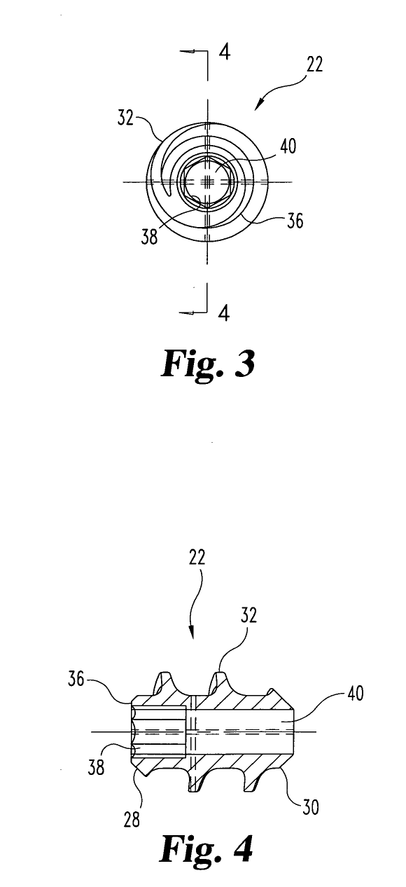

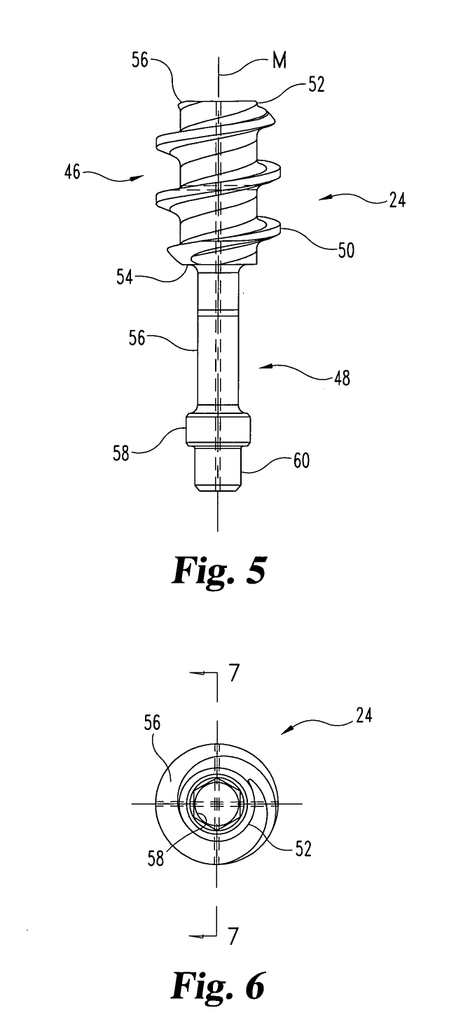

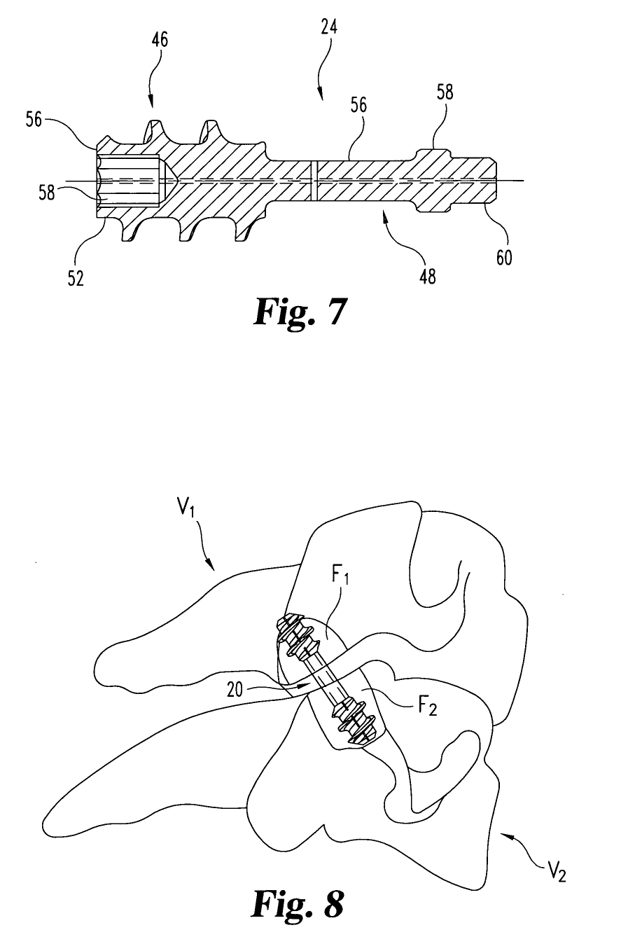

### DESCRIPTION OF THE ILLUSTRATED EMBODIMENTS

For the purposes of promoting an understanding of the principles of the disclosure, reference will now be made to the embodiment illustrated in the drawings and specific language will be used to describe the same. It will nevertheless be understood that no limitation of the scope of the claims is thereby intended, such alterations and further modifications in the illustrated device, and such further applications of the principles of the disclosure as illustrated therein, being contemplated as would normally occur to one skilled in the art to which the disclosure relates.

Referring generally to the figures, there is shown an embodiment of an orthopedic implant 20. The illustrated embodiment of implant 20 includes a jack base 22 and a jacking segment 24. That embodiment of implant 20 also includes an intermediate spacer 26. Implant 20 may be used in spinal orthopedic surgery or other orthopedic situations where maintenance or adjustment of spacing between tissues is desired. In a particular usage, implant 20 allows for an adjustable range of space between vertebral processes and facets (e.g. spinous processes, transverse processes, articular processes, facet joints) or other bone or tissues within the body. Once placed, implant 20 may also preserve the range of motion and flexibility needed to accommodate movement between two or more structures or a structure and other tissue.

In the illustrated embodiment, jack base 22 has a head end 28, a foot end 30, and a thread 32 extending substantially from head end 28 to foot end 30. Foot end 30 may have a tapered surface 34, so as to ease insertion of base 22 when placing base 22 into a bone. In a particular embodiment, foot end 30 tapers from a first diameter of base 22 (corresponding to the trough of thread 32) at approximately a 45 degree angle to the longitudinal central axis L of base 22. Depending on the place and manner of insertion (e.g. density and sturdiness of bone structure, surrounding tissue types, likelihood of unintentional injury to surrounding structures), end 30 could have a sharply sloped point, or may be configured to be self-tapping.

Head end 28 may include a generally flat end surface or edge 36. In the illustrated embodiment, an internal print 38 is provided in end 28, which is hexagonal as shown, but may have a hexalobed or other configuration suitable for accommodating a turning tool (not shown). Below and communicating with print 38 is a cannula 40 that, in this embodiment, extends through base 22 to foot end 30. Cannula 40 along with print 38 may be considered a passage that allows base 22 to be slid into the body over a guide wire, tool, or other device.

Thread 32, in the illustrated embodiment, is a cancellous thread designed for attaching to a portion of a vertebra or other bony structure. As shown, thread 32 extends along most or all of the length of base 22, from head end 28 to foot end 30. The crest height and other features of thread 32 may be determined with a particular bone or location in mind. For example, for use in a lumbar vertebra, the thread crest may be somewhat larger in diameter, while for use in cervical or thoracic vertebrae the crest may have a relatively smaller diameter, but the windings of thread 32 may be closer together and/or have a smaller pitch. Further, thread 32 may be of other types, including reverse angle thread (e.g. one in which a pressure flank generally faces toward the longitudinal central axis of the shaft) or a machine thread. A machine thread is particularly useful in situations in which base 22 is to be connected to another implant device.

Jacking segment 24, in the illustrated embodiment, has a body 46 and a connecting shaft 48. As shown, this embodiment of body 46 is quite similar to the illustrated embodiment of base 22, having a generally cylindrical configuration with an external thread 50. The external diameter (e.g. root diameter) and characteristics of thread 50 are substantially identical to those of base 22 in this embodiment. Body 46 has a head end 52 and a foot end 54 adjoining shaft 48, with thread 50 extending substantially from head end 52 to foot end 54. Head end 52 may include a generally flat end surface or edge 56. In the illustrated embodiment, an internal print 58 is provided in end 52, which is hexagonal as shown, but may have a hexalobed or other configuration suitable for accommodating a turning tool (not shown).

Thread 50, in the illustrated embodiment, is a cancellous thread designed for attaching to a portion of a vertebra or other bony structure. Thread 50 extends along most or all of the length of body 46, from head end 52 to foot end 54. As noted above with respect to thread 32, the crest height and other features of thread 50 may be determined with a particular bone or location in mind. In a particular embodiment in which thread 32 of base 22 is a cancellous thread for anchoring in bone, thread 50 may be substantially identical in pitch and substantially identical or slightly larger in crest height as compared to thread 32. As will be explained further below, providing such identical threads allows segment 34 to be threaded through the same hole through which base 22 was threaded.

In the illustrated embodiment, shaft 48 extends from foot end 54, and may be integral or monolithic with body 46. Shaft 48 includes a medial portion 56, a flange portion 58 and a tip portion 60, each of which are substantially cylindrical in the illustrated embodiment. Medial portion 56 may be of varying diameter or width, depending on the strength and/or elastic qualities desired in implant 20, and its length may be chosen depending on the overall separation desired between body 46 and base 22, e.g. a shorter length for use at a thoracic or relatively higher level of the spine, and a longer lengths for use at a lumbar or relatively lower level of the spine. Flange portion 58 has a diameter or width somewhat larger than that of medial portion 56, and in a particular embodiment may be thought of as extending in a plane substantially perpendicular to the central longitudinal axis M of shaft 48 and/or of segment 24 as a whole. The diameter or width and thickness of flange portion 58 may be selected so that part or all of flange portion 58 can be inserted into print 38 of base 22, or so that flange portion does not enter print 38 but can rest against end surface 36 of base 22. Tip portion 60 has a diameter that is at least slightly smaller than the internal dimension of cannula 40 in base 22, and in the illustrated embodiment tip portion 60 has a width or diameter that is slightly larger than that of medial portion 56. In other embodiments, however, tip 60 may be about the same size as or somewhat smaller than medial portion 56, for ease of manufacture, for particular sturdiness or elasticity considerations, or for other reasons. Thus, shaft 48 can be chosen to be long enough to accommodate the distance desired between structures (e.g. vertebrae) including any relative movement between the structures.

The illustrated embodiment also includes spacer element 26, which is compressible in a particular embodiment. Spacer 26 is generally configured as a sleeve and is located between base 22 and jacking segment 24 as a separate component. Spacer 26 has a first end 64 which may engage foot end 30 of base 22, a second end 66 which may engage head end 52 of jacking segment 24, and a central through-hole 68. As shown, this embodiment of spacer 26 has a substantially uniform cylindrical exterior having a radius that is approximately equal to or perhaps slightly smaller than the diameters (or root-diameters) of base 22 and body 46 of jacking segment 24. Hole 68 has a width or diameter that, in certain embodiments, is at least as large as the width or diameter of medial portion 56 of shaft 48, and in particular embodiments may be about the same size or slightly larger than the diameter or width of flange portion 58 of shaft 48. A length of spacer 26 may be chosen to preserve a desired distance between base 22 and body 46 of segment 24. Thus, spacer 26 is sized and configured to be maintained between base 22 and body 46 of jacking segment 24. Spacer 26 can be made of a number of materials, and if it is desired for spacer 26 to have some compressibility, it can be made of materials having any of a variety of durometers. Spacer 26, if present, may be loosely placed between base 22 and body 46 of segment 24 so that shaft 48 extends through hole 68 of spacer 26. Alternatively, spacer 26 may be firmly attached to one or both of base 22 and segment 24, as with an adhesive, a joinder using heat or solvent, or by other means.

Spacer or sleeve member 26 may maintain inter-structure height and give structural integrity to implant 20 while allowing some range of movement in rotation (e.g. in a plane generally orthogonal to the longitudinal axis of implant 20) and possibly in compression and/or distraction, thus preserving at least an approximation if not a complete normal range of motion. For example, if implant 20 is placed between adjacent spinous processes, some degree of rotational and/or longitudinal motion would be enabled, while maintaining at least a minimum distance between inferior and superior spinous processes. If implant 20 is placed in a joint space, such as between an inferior articular facet of one vertebra and a superior articular facet of a second, adjacent vertebra, implant 20 would allow similar types or ranges of relative motion between the facets, and at least a minimum spacing between the facets would be maintained.

Implant 20 is constructed such that it may be secured in a bodily structure, will remain secured and can accommodate movement while maintaining at least a desired minimum spacing. In the illustrated embodiment, implant 20 is manufactured as three separate pieces—base 22, jacking segment 24, and spacer 26, of which segment 24 and spacer 26 are pre-assembled and sent to medical personnel in a package or kit with an appropriate base 22. In other embodiments, a kit for assembling one or more custom implants 20 may be provided, having for example a number of bases 22 of varying size, a number of jacking segments 24 having varying characteristics (as suggested herein) and/or a number of differently-sized spacers 26. Medical personnel, such as a nurse or surgeon, may choose the most appropriate base 22, jacking segment 24 and spacer 26 from the kit, i.e. those having the length, radius, material and/or other characteristics most suited to the surgical situation. Similarly, whether pre-assembled or provided as a kit, base 22 and jacking segment 24 may be used as an implant without spacer 26.

In use, as suggested above, implant 20 is inserted in and between two bony or other tissue portions. One embodiment of an implantation and use method is provided below in the context of insertion between two facets F₁, F₂ of adjacent vertebrae V₁, V₂ (FIG. 8). It will be seen that use in other parts of the body, or in the articular facets or other parts of the vertebrae, can be made using processes identical or substantially similar to those described below.

Implant 20 may be inserted in the following manner. The area where implant 20 will be inserted is prepared. A single stab or puncture entry, which may be relatively small or minimally-invasive, is made in this embodiment for each implant 20 to be placed. Of course, if other procedures are necessary, an open or other surgical approach may be used, and implant 20 may be placed prior to or after such other procedures, as the surgeon may deem appropriate. Once the stab wound is made and a path to adjacent facets is created, the bones V₁, V₂ are drilled and tapped, so that a single hole (which may be substantially straight) exists through both a facet F₁ (e.g. a superior facet) and a facet F₂ (e.g. an inferior facet) and perhaps tissue between them. Such drilling and tapping can be accomplished using a standard drill, bit, and/or tap, or similar tools, or tapping can be accomplished by using a self-tapping jack base 22. Thus, a threaded bore is created through facet F₁ and facet F₂, and any tissue in the space between them is moved aside or also has such a threaded hole through it. Jack base 22, if not used to drill through a first bone portion and into a second, is inserted. In general, base 22 is threaded through the hole in facet F₁, threaded or otherwise maneuvered between facets F₁ and F₂, and threaded into facet F₂. A turning tool (not shown) having a connection end compatible with print 38 of base 22 can be used to thread base 22 into the surgical hole. Base 22 is threaded to the end of the drilled hole in facet F₂, or to another depth considered appropriate by the surgeon.

Once base 22 is anchored in facet F₂, spacer 26, if not attached to either base 22 or jacking segment 24, may be inserted through the surgical hole, and is placed adjacent and/or secured to end surface 36 of base 22. Jacking segment 24 (or the combination of segment 24 and spacer 26) is inserted through the threaded surgical bore in facet F₁. Spacer 26 may be pushed through the surgical hole in the first process because its diameter, in the illustrated embodiment, is equal to or slightly less than the root diameter of base 22 and/or segment 24. Segment 24 is threaded into facet F₁, e.g. via a turning tool (not shown) with a connecting end compatible with print 58 of segment 24) so that its shaft 48 extends through spacer 26 and into print 38 and/or cannula 40 of base 22. Thus, depending on the relative diameters of the relevant portions of base 22 and segment 24, tip portion 60 enters print 38 and possibly cannula 40, flange portion 58 may enter print 38 or at least abut end surface 36, and spacer 26 may abut both end surface 36 of base 22 and end 54 of segment 24. The contact of at least segment 24 and base 22 should be observed by the surgeon as contact between the two parts is made, and that contact may be verified x-ray or other means.

Adjustment of implant 20 and enlargement of the space between the implanted facets can then be made by further turning of segment 24. Once contact is made between shaft 48 of segment 24 and base 22 (or among segment 24, spacer 26 and base 22), and since turning of segment 24 is not transmitted to base 22 fixed in the first vertebra, further turning of segment 24 continues to translate segment 24 with respect to the second vertebra. Because the distance between segment 24 and base 22 does not change, the result of such further turning is to move one vertebra (e.g. V₁) or vertebral part (e.g. facet F₁) further away from the other vertebra (e.g. V₂) or vertebral part (e.g. facet F₂). In other words, when base 22 and jacking segment 24 are in contact, continuing to screw segment 24 expands the distance between the facets as more of segment 24 is forced into or through the first facet and perhaps into an inter-facet space. This “jacking” action allows for an infinitely fine range of adjustment to the distance between facets, spinous processes, or other parts of vertebrae or other tissues, subject to the length of the parts of implant 20, using few parts of mechanical simplicity and few or no sharp edges or other potentially injurious features.

Once the implant has been successfully placed and adjustments have been made, the opening may be closed, or other procedures may be performed in the same area before closing the opening. This process may be repeated for other areas, as needed. For example, if bilateral symmetry of vertebrae or vertebral spacing is desired, two implants may be placed, one on either side of the spinous process, for example, in the left and right facet joints. This “jacking procedure” may be used to increase inter-vertebral space, open impeded or collapsed facet joint spacing, maintain intervertebral space when either a disc is no longer present or is sufficiently degenerated, or otherwise create desired spacing between adjacent vertebral or other orthopedic surfaces. Rotation of the parts of implant 20 with respect to each other substantially about the axis of shaft 48 is possible, as well as some lateral motion where the sizing of base 22 and segment 24 will permit, and some extension (and reciprocal compression) along the axis of shaft 48. In cases where extension between vertebrae is desired, the length of shaft 48 inserted into base 22 may be long enough so that at maximum extension, a portion of shaft 48 remains in base 22. Spacer 26 helps maintain a minimum spacing, and where spacer 26 is compressible or elastic, provides some counterforce to return implant 20 to its original, implanted state. In this way, implant 20 maintains distance between structures and allows relative motion, thereby allowing a patient to experience a more normal range of motion.

As described above, implant 20 is inserted first through a superior structure and then into an inferior structure. This description should not be seen as limiting insertion of implant 20. It is understood that insertion of components of implant 20 could be performed inversely to the procedure described (e.g. first through an inferior structure). It is also understood that alternative attachment structures or methods may be used, such as by stapling or by applying cement or adhesive to base 22 or in a hole in which base 22 is placed, so long as base 22 is firmly fixed to its structure so as to withstand the stresses inherent in continual movements. Further, embodiments of implant 20 without spacer 26, e.g. with only an embodiment of base 22 and an embodiment of segment 24, may be used. As already discussed, embodiments of implant 20 may be used between a variety of vertebral parts or other tissues.

Base 22 and segment 24 are preferably made of biocompatible materials such as stainless steel, titanium, certain ceramics or hard plastics, or other known biocompatible materials or their equivalents. As discussed above, spacer 26 may be made of non-compressible or compressible biocompatible materials. The use of implant 20 has been primarily described in the posterior spinal area; however, it may be used in anterior, in other joints, between other bony structures, or wherever two structures need to have a distance maintained and are amenable to placement of segment 24 and base 22. Implant 20 generally has a low profile and a long axis sized and shaped to accommodate the area in which it will be placed. The radius of implant 20 may also maintain a low profile, and, as mentioned above with respect to specific components of implant, may be fixed or variable to accommodate the flexibility and strength required by the area in which it is to be placed. Implant 20 is generally coaxially aligned when placed, but it will be seen that offset configurations could be employed. Cross-section of components of implant 20 have generally been shown as circular, though other shaped cross-sections for some or all components could be used, including but not limited to triangular, square, hexagonal, polygonal or irregularly shaped.

While the illustrated embodiments have been detailed in the drawings and foregoing description, the same is to be considered as illustrative and not restrictive in character, it being understood that only the preferred embodiment has been shown and described and that all changes and modifications that come within the spirit of the invention are desired to be protected. The articles “a”, “an”, “said” and “the” are not limited to a singular element, and include one or more such elements.

What is claimed is:

1. An adjustable medical implant for use between portions of adjacent vertebrae comprising:
a jack base including an externally threaded anchoring portion and a central longitudinal aperture, said anchoring portion adapted for insertion into at least a portion of a first vertebra; and
a jacking segment including a body portion and a shaft portion, said body portion having an externally threaded portion for insertion into at least a portion of a second vertebra adjacent said first vertebra so that said shaft portion is at least partially within said aperture of said base;
wherein said jacking segment can rotate independently of said jack base; and
wherein turning said jacking segment relative to said base operates to alter a distance between the first and second vertebrae.

2. The implant of claim 1 further comprising a spacer member located around said shaft and between said jacking segment and said base.

3. The implant of claim 2 wherein said spacer member is a compressible sleeve.

4. The implant of claim 2 wherein said spacer member is firmly attached to said body portion of said jacking segment or said base.

5. The implant of claim 3 wherein said spacer member is firmly attached to said base.

6. The implant of claim 1 wherein said shaft includes a flange portion that extends substantially perpendicular to the longitudinal axis of said shaft.

7. The implant of claim 6 wherein said flange abuts said base when a portion of said shaft is within said aperture of said base.

8. The implant of claim 1 wherein said body portion of said jacking segment has an internal driving print.

9. The implant of claim 1 wherein said aperture in said base extends through the entire length of said base, and a portion of said aperture has an internal driving print.

10. The implant of claim 1 wherein said shaft portion and said body portion of said jack segment are monolithic.

11. The implant of claim 1, wherein said external threads of said base and said jacking segment are substantially identical in configuration.

12. An adjustable implant for use between portions of adjacent vertebrae, comprising:
a jack base having an externally threaded anchoring portion adapted to engage at least a portion of a first vertebra;
a jacking segment having a body portion and a shaft portion, said body being externally threaded for engaging at least a portion of a second vertebra and having an internal driving print; and
a sleeve member having a first end and a second end;
wherein said base has a head surface abutting said sleeve member, and wherein said shaft portion of said jacking segment extends through said sleeve.

13. The implant of claim 12 further comprising an axial cannula in said base, said cannula including a driving print.

14. The implant of claim 13, wherein said cannula extends through the entire length of said base.

15. The implant of claim 13, wherein said shaft includes a flange portion extending substantially perpendicular to the longitudinal axis of said shaft.

16. The implant of claim 15, wherein said flange portion abuts said base when said shaft extends into said cannula of said base.

17. The implant of claim 12, wherein said sleeve member is compressible.

18. The implant of claim 12, wherein said sleeve member is firmly attached to one of said base and said jacking segment.

19. A method of inserting an adjustable implant between first and second adjacent vertebrae comprising:
accessing a patient's spine via a stab or puncture entry;
making a hole in and through a portion the first and second vertebrae via said entry;
inserting a jack base having an aperture through said hole in said first vertebra and into said hole in said second vertebra, so that said base is attached only to said second vertebra;
inserting a jacking segment including an externally threaded body portion and a shaft portion into at least a portion of said hole in said first vertebra so that said shaft portion enters said aperture in said base and contacts a portion of said base; and
turning said jacking segment with respect to said base, thereby changing the spacing between at least a portion of said vertebrae.

20. The method of claim 19, wherein said base includes a threaded exterior, and said base-inserting step includes threading said base through said hole in said first vertebra and into said hole in said second vertebra.

21. The method of claim 19, wherein a spacer element having a central aperture is connected to said jacking segment so that said shaft extends through said central aperture of said spacer element, and said jacking-segment-inserting step includes abutting a portion of said shaft with said base.

22. The method of claim 19, wherein a spacer element having a central aperture is connected to said base, and said jacking-segment-inserting step includes inserting said shaft through said central aperture of said spacer element.
